(19) United States
(12) Patent Application Publication
(10) Pub. No.: US 2019/0015416 A1
(43) Pub. Date: Jan. 17, 2019

(54) BTK INHIBITORS FOR TREATING NEUROBLASTOMA

(71) Applicant: The Chinese University of Hong Kong, Shatin (CN)

(72) Inventors: Hui ZHAO, Shatin (CN); Jianmin Sun, Yinchuan (CN)

(21) Appl. No.: 16/028,024

(22) Filed: Jul. 5, 2018

Related U.S. Application Data
(60) Provisional application No. 62/531,196, filed on Jul. 11, 2017.

(51) Int. Cl. A61K 31/519; C07K 16/40; C12N 15/113; A61K 39/395; A61K 31/4545; A61P 35/00; G01N 33/573

(52) U.S. Cl. CPC A61K 31/519; C07K 16/40; C12N 15/1137; C12N 2310/14; A61K 31/4545; A61P 35/00; G01N 33/573; A61K 39/3955

(57) ABSTRACT

The present invention provides a novel method, composition, and kit for treating neuroblastoma by way of the use of a BTK inhibitor. Also provided is a method for identifying a BTK inhibitor.

### RELATED APPLICATIONS

This application claims priority to U.S. Provisional Patent Application No. 62/531,196, filed Jul. 11, 2017, the contents of which are hereby incorporated by reference in the entirety for all purposes.

### BACKGROUND OF THE INVENTION

Neuroblastoma is the third most common childhood cancer after leukemia and brain cancers, and it is one of the leading causes of childhood death by cancers. It often occurs in autonomic nervous system and medulla of adrenal gland, both of which are derived from neural crest during embryonic development (Park J R et al. Hematology/oncology clinics of North America. 2010; 24(1):65-86.). Treatment of neuroblastoma still relies on the conventional therapeutic approach, and the prognosis of high risk neuroblastoma remains far from satisfactory (Louis C U et al. Annual review of medicine. 2015; 66(49-63).

Protein tyrosine kinases catalyze tyrosine phosphorylation, which regulates signal transduction pathways that govern cell survival, proliferation, differentiation and as such are tightly regulated. Genes that regulate extracellular growth, differentiation and developmental signals are often dysregulated in cancers.

Bruton's tyrosine kinase (BTK) is a member of the Tec family of non-receptor tyrosine kinases. It plays an essential role in the B-cell signaling pathway linking cell surface B-cell receptor (BCR) stimulation to downstream intracellular responses (Kurosaki, Curr Op Imm, 2000, 276-281; Schaeffer and Schwartzberg, Curr Op Imm 2000, 282-288). In addition, BTK is also involved in other signaling pathways, e.g., Toll like receptor (TLR) and cytokine receptor-mediated TNF-alpha production in macrophages, IgE receptor (FcepsilonRI) signaling in Mast cells, inhibition of Fas/APO-1 apoptotic signaling in B-lineage lymphoid cells, and collagen-stimulated platelet aggregation (Jeffries, et al., 2003, Journal of Biological Chemistry 278:26258-26264; N. J. Horwood, et al., 2003, the Journal of Experimental Medicine 197:1603-1611; Iwaki et al. 2005, Journal of Biological Chemistry 280(48):40261-40270; Vassilev et al. 1999, Journal of Biological Chemistry 274(3):1646-1656, and Quek et al. 1998, Current Biology 8(20):1137-1140).

Because of the high prevalence of neuroblastoma and its significant impact on human health, there exists an urgent need for new and more effective methods to treat neuroblastoma. This invention fulfills this and other related needs.

### BRIEF SUMMARY OF THE INVENTION

The present inventors have identified Bruton's tyrosine kinase (BTK) as a novel therapeutic target for human neuroblastoma. More specifically, the inventors observed in their studies that, BTK interacts with Anaplastic lymphoma kinase (ALK) and activates downstream kinases such as ERK in neuroplastoma cells. On the other hand, suppression of BTK activity by a small molecule inhibitor has been shown to inhibit the growth and survival of neuroblastoma cells and induces their programmed cell death.

As such, in the first aspect, the present invention provides a method for treating neuroblastoma. The method includes the step of administering to a subject in need thereof an effective amount of a Bruton's tyrosine kinase (BTK) inhibitor. In some embodiments, the BKT inhibitor is 1-((R)-3-(4-amino-3-(4-phenoxyphenyl)-1H-pyrazolo[3,4-d]pyrimidin-1-yl)pi-peridin-1-yl)prop-2-en-1-one (Ibrutinib). In some embodiments, the BKT inhibitor is a neutralizing antibody of BTK. In some embodiments, the BKT inhibitor is an antisense oligonucleotide or siRNA that suppresses BTK expression. In some embodiments, the subject is co-administered with a second therapeutic agent for treating neuroblastoma, such as an ALK inhibitor, e.g., Crizotinib. In some embodiments, the subject has wild-type ALK gene. In other embodiments, the subject has a mutated ALK gene or ALK overexpression or ALK aberrant activation.

In a related aspect, the present invention provides the novel use of a BTK inhibitor for the manufacturing of a medicament for treating neuroblastoma. In some embodiments, the BTK inhibitor is 1-((R)-3-(4-amino-3-(4-phenoxyphenyl)-1H-pyrazolo[3,4-d]pyrimidin-1-yl)pi-peridin-1-yl)prop-2-en-1-one (Ibrutinib). In some embodiments, the BKT inhibitor is a neutralizing antibody of BTK. In some embodiments, the BTK inhibitor is an antisense oligonucleotide or siRNA that suppresses BTK expression. In some embodiments, a second therapeutic agent for treating neuroblastoma is used with the BTK inhibitor for making the medicament. The second therapeutic agent may be an ALK inhibitor, e.g., Crizotinib.

In a second aspect, the present invention provides a composition comprising a BTK inhibitor intended for the treatment of neuroblastoma. The composition includes an effective amount of a BTK inhibitor and a physiologically acceptable excipient. In some embodiments, the inhibitor is 1-((R)-3-(4-amino-3-(4-phenoxyphenyl)-1H-pyrazolo[3,4-d]pyrimidin-1-yl)pi-peridin-1-yl)prop-2-en-1-one (Ibrutinib); or a neutralizing antibody of BTK; or an antisense oligonucleotide or siRNA that suppresses BTK expression. In some embodiments, the composition further comprises an ALK inhibitor such as Crizotinib.

In a third aspect, the present invention provides a method for identifying a BTK inhibitor. The method includes these steps: (a) contacting a cell expressing both BTK and ALK with a candidate compound; (b) determining BTK-ALK association level in the cell in step (a); (c) comparing the BTK-ALK associate level obtained in step (b) with a control BTK-ALK association level in a control cell, which is identical to the cell in step (a) but has not been contacted with the candidate compound; and (d) identifying the candidate compound as a BTK inhibitor, when the BTK-ALK associate level obtained in step (b) is lower than the control BTK-ALK association level. In some embodiments, the BTK-ALK associate level obtained in step (b) is at least 10%, 20%, or 50% lower than the control BTK-ALK association level. In some embodiments, the cell is a neuroblast. In some embodiments, this screening method also includes the additional steps, subsequent to step (d), of: contacting neuroblastoma cells with the candidate compound and measuring proliferation rate or apoptosis rate of the cells.

In a fourth aspect, this invention provides a kit for treating neuroblastoma in a subject. The kit includes a first container containing a BTK inhibitor and a second container containing a second therapeutic agent for treating neuroblastoma. In some embodiments, the BTK inhibitor is 1-((R)-3-(4-amino-3-(4-phenoxyphenyl)-1H-pyrazolo[3,4-d]pyrimidin-1-yl)pi-peridin-1-yl)prop-2-en-1-one (Ibrutinib); or a neutralizing antibody of BTK; or an antisense oligonucleotide or siRNA that suppresses BTK expression. In some embodiments, the second therapeutic agent is an ALK inhibitor, such as Crizotinib. In some embodiments, the kit also includes an instruction manual to provide information for a user to use the kit.

### BRIEF DESCRIPTION OF THE DRAWINGS

FIGS. 1A-1C: BTK physically interacts with ALK. (FIG. 1A) Co-immunoprecipitation (Co-IP) indicated the physical binding between the ALK and BTK. COS1 cells were transfected with either ALK^{WT}, BTK separately or both. Cell lysates were pulled down by ALK antibody, and the precipitates were detected by indicated antibodies. (FIG. 1B) Co-IP reveals the binding between the endogenous BTK and ALK. The cell lysates from NBL-S or SH-SY5Y cells were immunoprecipited with ALK antibody or mouse IgG, and then detected by ALK antibody and BTK antibody, respectively. (FIG. 1C) Either ALK^{WT} or ALK^{F1174L} were transfected into COS1 cells with or without BTK. After cell starvation and stimulation with mAb16-39, cells were lysed, and the lysates were precipitated with anti-Flag antibody, followed by separation by SDS-PAGE. ALK antibody, 4G10, BTK antibody and TUBULIN antibody were used to probe the membrane for immunoprecipitats and whole cell lysate, respectively.

FIGS. 2A-2C: BTK is expressed in neuroblastoma tumors and cell lines. (FIGS. 2A, 2B) BTK expression in neuroblastoma cell lines revealed by Western blot (FIG. 2C) and RT-PCR (D), respectively. (FIG. 2C) BTK was expressed in human neuroblastoma tumor tissues revealed by DAB staining. The scale bar is 100 μm.

FIG. 3: ALK^{F1174L} enhances phosphorylation of BTK in neuroblastoma cell. ALK^{WT} or ALK^{F1174L} was overexpressed in NBL-S with or without BTK. The total BTK and ALKs, as well as the pBTK and pALK were detected by indicated antibodies.

FIGS. 4A-4B: Ibrutinib inhibits phosphorylation of ERK in neuroblastomas cells. NBL-S cells (FIG. 4A) and SH-SY5Y (FIG. 4B) cells were starved in medium without serum for 4 hours and treated with Iburutinib (2.5 μM), Crizotinib (0.25 μM), TAE684 (0.1 μM), Iburutinib (2.5 μM)+Crizotinib (0.25 μM) or Iburutinib (0.25 μM)+TAE684 (0.25 μM) during the starvation. Cell lysate was probed with indicated antibody respectively.

FIGS. 5A-5G: BTK signaling regulates cell proliferation. (FIGS. 5A, 5B) Ibrutinib treatment attenuated proliferation of neuroblastoma cells. NBL-S cells (FIG. 5A) and SH-SY5Y cells (FIG. 5B) were treated with Ibrutinib, Crizotinib, NVP-TAE684 separately for 48 hr. MTT were performed to measure the cell viability, the absorbance of treated cell groups was normalized to that of DMSO treated cells. (FIGS. 5C, 5D) MTT assay of NBL-S or SH-SY5Y cells transfected with BTK (FIG. 5C) or BTK siRNA (FIG. 5D) at 48 hr after transfection. Overexpression of BTK increases, while knockdown of BTK attenuates cell proliferation. (FIGS. 5E, 5F) NBL-S cells (FIG. 5E) and SH-SY5Y (FIG. 5F) cells were treated with either Ibrutinib, Crizotinib, NVP-TAE684 separately or with combinations as indicated for 48 hr, followed by measurement with MTT assay. (FIG. 5E) NBL-S cells and SH-SY5Y cells were treated with the inhibitors either separately or the combinations as indicated, and the treated cells were collected at different time points for MTT assay. The absorbance of treated cell groups was normalized to that of control cells treated with DMSO.

FIGS. 6A-6D: Ibrutinib treatment induced cell cycle arrest and promoted cell apoptosis of neuroblastoma cells. (FIG. 6A) Cell cycle distribution of SH-SY5Y cells after treatment with NVP-TAE684 (100 nM), Ibrutinib (2.5 μM, 10 μM) and Crizotinib (250 nM) for 24 hr. Representative graph from three repeat experiments are shown. (FIG. 6B) Ibrutinib (2.5 μM) increases the effects of NVP-TAE684 (100 nM) and Crizotinib (250 nM) on cell cycle arresting at G0/G1 phase in neuroblastoma cells. The combined treatment with Ibrutinib and NVP-TAE684 induced a further increase of cell portion at G0/G1 phase to 78.34%±0.7% (p<0.01), compared with the control (64.90%±0.86%) and the single inhibitor treatment, e.g., Ibrutinib (73.75%±1.2%), NVP-TAE684 (74.48%±0.07%) and Crizotinib (68.10%±0.66%). (FIG. 6C) The cell apoptosis rates were measured by flow cytometry 48 hr after the treatment of indicated inhibitors. The apoptosis rates are 18.8%±2.26% induced by Ibrutinib (2.5 μM), 18.3%±0.42% by Crizotinib (250 nM), and 25.85%±1.48% for NVP-TAE684 (100 nM). The control cells were treated with DMSO showing 14.05%±2.19% of apoptosis rate. (FIGS. 6D, 6E) Western blot showing apoptosis markers in NBL-S (FIG. 6D) and SH-SY5Y (FIG. 6E) cells treated with the indicated inhibitors and inhibitor combinations. Red arrows mark the cleaved PARP.

FIGS. 7A-7H: Ibrutinib inhibits tumorigenicity of SH-SY5Y in nude mice. SH-SY 5Y cells were inoculated into flanks of nude mice. Animals were randomly divided into four groups when tumor xenograft reached 100 mm³. Vehicle, Ibrutinib, Crizotinib, and the combination were respectively administered to treat the animals for 14 days. (FIG. 7A) Photographs of xenografts dissected from the nude mice at day 14 of treatment. (FIG. 7B) Quantifications of the tumor xenografts weight. The data are expressed as mean values±S.D. (n=4 in each group). **P<0.01. (FIGS. 7C, C′) Growth curve of xenograft tumor volumes of in nude mice treated the inhibitors for 14 days. Mean±SD values are presented. **p<0.01; *p<0.05. The tumor growth in nude mice treated with Crizotinib and the combination of Crizotinib and Ibrutinib was highlighted in (FIG. 7C′). (FIG. 7D) Cell proliferations in tumor xenografts were elucidated by immunohistochemical (IHC) analysis with Ki67 antibody. Scale bar represents 50 μm. (FIG. 7E) Quantification of Ki-67-positive cells within the xenografts. Ki-67-positive cells were quantitated from 5 random fields of each tumor xenograft under the microscope (400×) using Image-Pro PLUS V6.0.0.260 software. The values were normalized to those from vehicle treated mice. The data were presented as mean values±S.D. (n=4). **p<0.01; *p<0.05. Apoptosis markers such as cleaved PARP and cleaved Caspase 3 in xenograft tumors were examined by Western blot. (FIG. 7F) The representative gel showing the expression of apoptosis maker from two xenograft tumors from each treatment group. The quantifications of Western blot were shown in (FIG. 7G) and (FIG. 7H) for cleaved PARP and cleaved Caspase 3. The signals densities were analyzed by Image J. The relative signals of cleavage PARP to total PARP, cleaved Caspase 3 to tubulin were normalized to those from vehicle treated mice. The ratios indicate the increase of apoptosis.

### DEFINITIONS

The term “BTK” or “Bruton's tyrosine kinase,” as used herein, refers to a tyrosine kinase that is a key component of B cell receptor (BCR) signalling and functions as an important regulator of cell proliferation and cell survival in various B cell malignancies such as lymphoma. It is encoded by the BTK gene located on human X chromosome. The GenBank Accession Nos. are NM_000061, NM_001287344, and NM_001287345 for human ALK mRNA and NP_000052, NP_001274273, and NP_001274274 for human ALK protein, respectively. The term “ALK” or “Anaplastic lymphoma kinase,” as used herein, refers to a kinase also known as ALK tyrosine kinase receptor or CD246 (cluster of differentiation 246), an enzyme that in humans is encoded by the ALK gene located on chromosome No. 2. The GenBank Accession Nos are NM_004304 for human ALK mRNA and NP_004295 for human ALK protein, respectively.

In this disclosure the term “neuroblastoma” refers to a type of cancer that frequently begins in certain very early forms of nerve cells (called neuroblasts) found in the sympathetic nervous system of an embryo or fetus. This type of cancer occurs most often in infants and young children, and it is rarely found in children older than 10 years. More than 1 out of 3 neuroblastomas start in the adrenal glands, whereas about 1 out of 4 begin in sympathetic nerve ganglia in the abdomen. Most of the rest start in sympathetic ganglia near the spine in the chest or neck, or in the pelvis. The progression of neuroblastomas can vary significantly: some grow and spread quickly, while others grow slowly. Sometimes, in very young children, the cancer cells die for no reason and the tumor goes away on its own. In other cases, the tumor cells mature on their own into normal ganglion cells and stop dividing.

In this disclosure the term “or” is generally employed in its sense including “and/or” unless the content clearly dictates otherwise.

As used herein, the term “gene expression” is used to refer to the transcription of a DNA to form an RNA molecule encoding a particular protein (e.g., human BTK protein) or the translation of a protein encoded by a polynucleotide sequence. In other words, both mRNA level and protein level encoded by a gene of interest (e.g., human BTK gene) are encompassed by the term “gene expression level” in this disclosure.

In this disclosure the term “biological sample” or “sample” includes sections of tissues such as biopsy and autopsy samples, and frozen sections taken for histologic purposes, or processed forms of any of such samples. Biological samples include blood and blood fractions or products (e.g., serum, plasma, platelets, red blood cells, white blood cells, and the like), sputum or saliva, lymph and tongue tissue, cultured cells, e.g., primary cultures, explants, and transformed cells, stool, urine, colon biopsy tissue etc. A biological sample is typically obtained from a eukaryotic organism, which may be a mammal, may be a primate and may be a human subject.

In this disclosure the term “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., whole blood, blood cells such as red or white blood cells, plasma/serum, lymph nodes, liver, bone marrow, spleen, etc.) among other factors. Representative biopsy techniques include, but are not limited to, excisional biopsy, incisional biopsy, needle biopsy, surgical biopsy, and bone marrow biopsy and may comprise blood drawing. A wide range of biopsy techniques are well known to those skilled in the art who will choose between them and implement them with minimal experimentation.

In this disclosure the term “isolated” nucleic acid molecule means a nucleic acid molecule that is separated from other nucleic acid molecules that are usually associated with the isolated nucleic acid molecule. Thus, an “isolated” nucleic acid molecule includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose, or polyacrylamine) containing restriction-digested genomic DNA, is not an “isolated” nucleic acid.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) involved in the transcription/translation of the gene product and regulation of the transcription/translation, as well as intervening sequences (introns) between individual coding segments (exons).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. For the purposes of this application, amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. For the purposes of this application, amino acid mimetics refers to chemical compounds that have a structure that is different from the general chemical structure of an amino acid, but that functions in a manner similar to a naturally occurring amino acid.

Amino acids may include those having non-naturally occurring D-chirality, as disclosed in WO01/12654, which may improve the stability (e.g., half-life), bioavailability, and other characteristics of a polypeptide comprising one or more of such D-amino acids. In some cases, one or more, and potentially all of the amino acids of a therapeutic polypeptide have D-chirality.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. “Operably linked” in this context means two or more genetic elements, such as a polynucleotide coding sequence and a promoter, placed in relative positions that permit the proper biological functioning of the elements, such as the promoter directing transcription of the coding sequence. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

The term “immunoglobulin” or “antibody” (used interchangeably herein) refers to an antigen-binding protein having a basic four-polypeptide chain structure consisting of two heavy and two light chains, said chains being stabilized, for example, by interchain disulfide bonds, which has the ability to specifically bind antigen. Both heavy and light chains are folded into domains.

The term “antibody” also refers to antigen- and epitope-binding fragments of antibodies, e.g., Fab fragments, that can be used in immunological affinity assays. There are a number of well characterized antibody fragments. Thus, for example, pepsin digests an antibody C-terminal to the disulfide linkages in the hinge region to produce F(ab)′₂, a dimer of Fab which itself is a light chain joined to V_{H}-C_{H}1 by a disulfide bond. The F(ab)′₂ can be reduced under mild conditions to break the disulfide linkage in the hinge region thereby converting the (Fab′)₂ dimer into an Fab′ monomer. The Fab′ monomer is essentially a Fab with part of the hinge region (see, e.g., Fundamental Immunology, Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody also includes antibody fragments either produced by the modification of whole antibodies or synthesized using recombinant DNA methodologies.

The phrase “specifically binds,” when used in the context of describing a binding relationship of a particular molecule (e.g., an anti-BTK antibody) to a protein or peptide (e.g., a human BTK protein), refers to a binding reaction that is determinative of the presence of the protein in a heterogeneous population of proteins and other biologics. Thus, under designated binding assay conditions, the specified binding agent (e.g., an antibody) binds to a particular protein at least two times the background and does not substantially bind in a significant amount to other proteins present in the sample. Specific binding of an antibody under such conditions may require an antibody that is selected for its specificity for a particular protein or a protein but not its similar “sister” proteins. A variety of immunoassay formats may be used to select antibodies specifically immunoreactive with a particular protein or in a particular form. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective binding reaction will be at least twice background signal or noise and more typically more than 10 to 100 times background. On the other hand, the term “specifically bind” when used in the context of referring to a polynucleotide sequence forming a double-stranded complex with another polynucleotide sequence describes “polynucleotide hybridization” based on the Watson-Crick base-pairing, as provided in the definition for the term “polynucleotide hybridization method.”

As used in this application, an “increase” or a “decrease” refers to a detectable positive or negative change in quantity from a comparison control, e.g., an established standard control (such as an expression level of BTK mRNA or protein). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

The term “inhibiting” or “inhibition,” as used herein, refers to any detectable negative effect on a target biological process, such as mRNA/protein expression, protein interaction (e.g., between BTK and ALK) cellular signal transduction (e.g., ERK activation mediated by BTK), cell proliferation, tumorigenicity, metastatic potential, and recurrence of a disease/condition. Typically, an inhibition is reflected in a decrease of at least 10%, 20%, 30%, 40%, or 50% in target process (e.g., expression of BTK at either mRNA level or protein level) upon application of an inhibitor, when compared to a control where the inhibitor is not applied.

The term “amount” as used in this application refers to the quantity of a polynucleotide of interest or a polypeptide of interest, e.g., human BTK mRNA or protein, present in a sample. Such quantity may be expressed in the absolute terms, i.e., the total quantity of the polynucleotide or polypeptide in the sample, or in the relative terms, i.e., the concentration of the polynucleotide or polypeptide in the sample.

The term “treat” or “treating,” as used in this application, describes to an act that leads to the elimination, reduction, alleviation, reversal, or prevention or delay of onset or recurrence of any symptom of a relevant condition. In other words, “treating” a condition encompasses both therapeutic and prophylactic intervention against the condition.

The term “effective amount” as used herein refers to an amount of a given substance that is sufficient in quantity to produce a desired effect. For example, an effective amount of an inhibitor of BTK is the amount of said inhibitor to achieve a decreased level of BTK mRNA or protein expression or biological activity including reduced interaction with ALK and/or reduced activation of ERK, such that the symptoms, severity, and/or recurrence change of neuroblastoma are reduced, reversed, eliminated, prevented, or delayed of the onset in a patient who has been given the inhibitor for therapeutic purposes. An amount adequate to accomplish this is defined as the “therapeutically effective dose.” The dosing range varies with the nature of the therapeutic agent being administered and other factors such as the route of administration and the severity of a patient's condition.

The term “subject” or “subject in need of treatment,” as used herein, includes individuals who seek medical attention due to risk of, or actual suffering from, or risk of recurrence of, neuroblastoma. Subjects also include individuals currently undergoing therapy that seek manipulation of the therapeutic regimen. Subjects or individuals in need of treatment include those that demonstrate symptoms of neuroblastoma or are at risk of suffering from and/or recurrence of neuroblastoma or its symptoms. For example, a subject in need of treatment includes individuals with a genetic predisposition or family history for neuroblastoma, those that have suffered relevant symptoms in the past, those that have been exposed to a triggering substance or event, as well as those suffering from chronic or acute symptoms of the condition. A “subject in need of treatment” may be at any age of life, although pediatric patients are disproportionally affected by neuroblastoma.

“Inhibitors,” “activators,” and “modulators” of BTK are used to refer to inhibitory, activating, or modulating molecules, respectively, identified using in vitro and in vivo assays for BTK protein binding or signaling, e.g., ligands, agonists, antagonists, and their homologs and mimetics. The term “modulator” includes inhibitors and activators. Inhibitors are agents that, e.g., partially or totally block binding, decrease, prevent, delay activation, inactivate, desensitize, or down regulate the activity of BTK protein. In some cases, the inhibitor directly or indirectly binds to the BTK protein, such as a neutralizing antibody, such that it interferes with and suppresses BTK interaction with ALK, or it interferes with and suppresses BTK-mediated ERK activation. Inhibitors, as used herein, are synonymous with inactivators and antagonists. Activators are agents that, e.g., stimulate, increase, facilitate, enhance activation, sensitize or up regulate the expression of the BTK mRNA or protein and/or activity of the BTK protein. Modulators include BTK protein ligands or binding partners, including modifications of naturally-occurring ligands and synthetically-designed ligands, antibodies and antibody fragments, antagonists, agonists, small molecules including carbohydrate-containing molecules, siRNAs, RNA aptamers, and the like.

### DETAILED DESCRIPTION OF THE INVENTION

### I. Introduction

This invention relates generally to the use of Bruton's tyrosine kinase (BTK) inhibitors for the treatment of patients who suffer from neuroblastoma. Neuroblastoma is the third most common childhood cancer after leukemia and brain cancers, and it is one of the leading causes of childhood death by cancers. It often occurs in autonomic nervous system and medulla of adrenal gland, both of which are derived from neural crest during embryonic development (1). Treatment of neuroblastoma still relies on the conventional therapeutic approach, and the prognosis of high risk neuroblastoma remains far from satisfactory (2).

Bruton's tyrosine kinase (BTK) is a member of the Tec family of non-receptor tyrosine kinases. It plays an essential role in the B-cell signaling pathway linking cell surface B-cell receptor (BCR) stimulation to downstream intracellular responses (4). In addition, BTK is also involved in other signaling pathways, e.g., Toll like receptor (TLR) and cytokine receptor-mediated TNF-alpha production in macrophages, IgE receptor (FcepsilonRI) signaling in Mast cells, inhibition of Fas/APO-1 apoptotic signaling in B-lineage lymphoid cells, and collagen-stimulated platelet aggregation (5, 8-10).

The present inventors discovered of BTK expression, its interaction with ALK and its contribution of ERK activation in neuroblastoma. Described herein is the Bruton's tyrosine kinase (BTK) inhibitor, 1-((R)-3-(4-amino-3-(4-phenoxyphenyl)-1H-pyrazolo[3,4-d]pyrimidin-1-yl)pi-peridin-1-yl)prop-2-en-1-one (Ibrutinib), pharmaceutical formulations thereof, as well as pharmaceutical compositions that comprise the BTK inhibitor and methods of using the BTK inhibitor in the treatment of diseases or conditions that would benefit from inhibition of BTK activity, such as neuroblastoma.

### II. General Methodology

Practicing this invention utilizes routine techniques in the field of molecular biology. Practicing this invention utilizes routine techniques in the field of molecular biology. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)). Specifically Western blot, RT-PCR, MTT assay, and cell flow cytometry were performed as previously published (11, 12).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

The sequence of interest used in this invention, e.g., the polynucleotide sequence of the human BTK gene, and synthetic oligonucleotides (e.g., primers) can be verified using, e.g., the chain termination method for double-stranded templates of Wallace et al., Gene 16: 21-26 (1981).

### III. Treatment of Neuroblastoma

By illustrating the role of BTK in neuroblastoma, especially its interaction with Anaplastic lymphoma kinase (ALK) and activation of ERK, the present invention further provides a means for treating patients suffering from neuroblastoma: by way of administration of a BTK inhibitor to a neuroblastoma patient to suppress BTK mRNA or protein expression or inhibit BTK protein's biological activity. Some exemplary BTK inhibitors include, but are not limited to, first generation inhibitors such as 1-((R)-3-(4-amino-3-(4-phenoxyphenyl)-1H-pyrazolo[3,4-d]pyrimidin-1-yl)pi-peridin-1-yl)prop-2-en-1-one (Ibrutinib) and second generation inhibitors such as ACP-196 (acalabrutinib), ONO/GS-4059, and BGB-3111 (13), RN468 and CC-292 (AVL-292)(14, 15).

Neuroblastoma is a type of cancer that forms in certain types of nerve tissue. It most frequently starts from one of the adrenal glands, but can also develop in the neck, chest, abdomen, or spine. At early stages of the disease, symptoms of neuroblastoma are often vague, making diagnosis difficult. Fatigue, loss of appetite, fever, and joint pain are common. Additional symptoms may include bone pain, a lump in the abdomen, neck, or chest, or a painless bluish lump under the skin, and they may vary depending on primary tumor locations and metastases if present. Aside from these symptoms, the diagnosis of neuroblastoma is usually confirmed by a surgical pathologist, taking into account the clinical presentation, microscopic findings, and other laboratory tests.

The therapies for neuroblastoma also vary depending on the stage of the disease: earlier stages or low-risk disease can frequently be cured with surgery alone; whereas later stages or higher risk disease is treated with surgery, chemotherapy, radiation therapy, bone marrow transplantation, hematopoietic stem cell transplantation, biological-based therapy with 13-cis-retinoic acid (isotretinoin or Accutane), or antibody therapy (often co-administered with the cytokines GM-CSF and IL-2), or any combination thereof. Chemotherapy agents used in combination have been found to be effective against neuroblastoma. Agents commonly used in induction and for stem cell transplant conditioning are platinum compounds (cisplatin, carboplatin), alkylating agents (cyclophosphamide, ifosfamide, melphalan), topoisomerase II inhibitor (etoposide), anthracycline antibiotics (doxorubicin) and vinca alkaloids (vincristine), and topoisomerase I inhibitors (topotecan and irinotecan). Any of these agents or combinations thereof may be used in co-administration with a BTK inhibitor in the practice of the treatment method of this invention.

### A. Suppressing BTK Expression or Activity

1. Inhibitors of BTK mRNA

Suppression of BTK expression can be achieved through the use of nucleic acids siRNA, microRNA, miniRNa, lncRNA, antisense oligonucleotides, aptamer. Such nucleic acids can be single-stranded nucleic acids (such as mRNA) or double-stranded nucleic acids (such as DNA) that can translate into an active form of inhibitor of BTK mRNA under appropriate conditions.

In one embodiment, the BTK inhibitor-encoding nucleic acid is provided in the form of an expression cassette, typically recombinantly produced, having a promoter operably linked to the polynucleotide sequence encoding the inhibitor. In some cases, the promoter is a universal promoter that directs gene expression in all or most tissue types; in other cases, the promoter is one that directs gene expression specifically in neuroblast cells, especially in neuroblastoma cells. Administration of such nucleic acids can suppress BTK expression in the target tissue, e.g., neuroblastoma cells. Since the human BTK gene sequence encoding its mRNA is known as GenBank Accession Nos. NM_000061, NM_001287344, and NM_001287345, one can devise a suitable BTK-suppressing nucleic acid from the sequence, species homologs, and variants of these sequences.

### 2. Inhibitors of BTK Protein

Suppression of BTK protein activity can be achieved with an agent that is capable of inhibiting the activity of BTK protein. Since BTK, a tyrosine kinase, has now been shown to interact with ALK and to activate ERK, an in vitro assay or cell-based assay can be used to screen for potential inhibitors of ALK protein activity based on inhibited/abolished binding between BTK protein and ALK protein or reduced/abolished ERK activation mediated by BTK when a candidate compound is present. Once a compound is identified in the binding assay or ERK activation assay, further testing may be conducted to confirm and verify the compound's capability to inhibit BTK protein activity, its capability to suppress ERK activation, and/or its capability to inhibit neuroblastoma cell proliferation or survival rate. In general, such an assay can be performed in the presence of BTK protein or a fragment thereof, for example, a recombinantly produced BTK protein or fragment, under conditions permitting its binding to ALK. For convenience, the BTK protein or the candidate compound may be immobilized onto a solid support and/or labeled with a detectable moiety. A third molecule, such as an antibody (which may include a detectable label) to BTK protein, can also be used to facilitate detection.

In some cases, the binding assays can be performed in a cell-free environment; whereas in other cases, the binding assays can be performed within a cell such as a neuroblast or neuroblastoma cell, for example, using cells recombinantly or endogenously expressing the appropriate BTK polypeptide and ALK protein.

The anti-neuroblastoma effects of a BTK protein inhibitor of the present invention can also be demonstrated in in vivo assays. For example, a BTK protein inhibitor can be injected into animals that have a compromised immune system (e.g., nude mice, SCID mice, or NOD/SCID mice) and therefore permit xenograft neuroblastomas. Injection methods can be subcutaneous, intramuscular, intravenous, intraperitoneal, or intratumoral in nature. Cancer development is subsequently monitored by various means, such as measuring cancer cell proliferation and disease relapse/recurrence, in comparison with a control group of animals with the same or similar disease but not given the inhibitor. An inhibitory effect is detected when a negative effect on neuroblastoma cell proliferation or disease recurrence is established in the test group. Preferably, the negative effect is at least a 10% decrease; more preferably, the decrease is at least 20%, 30%, 40%, 50%, 60%, 70%, 80%, or 90%.

As stated above, BTK protein inhibitors can have diverse chemical and structural features. For instance, an inhibitor can be a non-functional BTK protein mutant that retaining the binding ability of wild-type BTK protein to ALK, an antibody to the BTK protein that interferes with BTK protein activity (e.g., a neutralizing antibody), or any small molecule or macromolecule that simply hinders the interaction between BTK protein and ALK protein. Essentially any chemical compound can be tested as a potential inhibitor of BTK protein activity. Most preferred are generally compounds that can be dissolved in aqueous or organic (especially DMSO-based) solutions. Inhibitors can be identified by screening a combinatorial library containing a large number of potentially effective compounds. Such combinatorial chemical libraries can be screened in one or more assays, as described herein, to identify those library members (particular chemical species or subclasses) that display a desired characteristic activity. The compounds thus identified can serve as conventional “lead compounds” or can themselves be used as potential or actual therapeutics.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)) and carbohydrate libraries (see, e.g., Liang et al., Science, 274:1520-1522 (1996) and U.S. Pat. No. 5,593,853). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and benzodiazepines, U.S. Pat. No. 5,288,514).

### B. Pharmaceutical Compositions

### 1. Formulations

Compounds of the present invention, such as BTK inhibitors, are useful in the manufacture of a pharmaceutical composition or a medicament. A pharmaceutical composition or medicament can be administered to a subject for the treatment of neuroblastoma.

Compounds used in the present invention, e.g., an inhibitor of BTK mRNA or protein (e.g., a neutralizing antibody against BTK protein), a nucleic acid encoding a polynucleotide or polypeptide inhibitor for BTK gene expression or BTK protein activity (e.g., an expression vector encoding a neutralizing antibody against BTK protein), are useful in the manufacture of a pharmaceutical composition or a medicament comprising an effective amount thereof in conjunction or mixture with excipients or carriers suitable for application.

An exemplary pharmaceutical composition for suppressing BTK expression comprises (i) an express cassette comprising a polynucleotide sequence encoding an inhibitor of BTK protein as described herein, and (ii) a pharmaceutically acceptable excipient or carrier. The terms pharmaceutically-acceptable and physiologically-acceptable are used synonymously herein. The expression cassette may be provided in a therapeutically effective dose for use in a method for treatment as described herein.

A BTK inhibitor or a nucleic acid encoding a BTK inhibitor can be administered via liposomes, which serve to target the conjugates to a particular tissue, as well as increase the half-life of the composition. Liposomes include emulsions, foams, micelles, insoluble monolayers, liquid crystals, phospholipid dispersions, lamellar layers and the like. In these preparations the inhibitor to be delivered is incorporated as part of a liposome, alone or in conjunction with a molecule which binds to, e.g., a receptor prevalent among the targeted cells (e.g., lymphoblasts), or with other therapeutic or immunogenic compositions. Thus, liposomes filled with a desired inhibitor of the invention can be directed to the site of treatment, where the liposomes then deliver the selected inhibitor compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al. (1980) Ann. Rev. Biophys. Bioeng. 9: 467, U.S. Pat. Nos. 4,235,871, 4,501,728 and 4,837,028.

Pharmaceutical compositions or medicaments for use in the present invention can be formulated by standard techniques using one or more physiologically acceptable carriers or excipients. Suitable pharmaceutical carriers are described herein and in “Remington's Pharmaceutical Sciences” by E. W. Martin. Compounds and agents of the present invention and their physiologically acceptable salts and solvates can be formulated for administration by any suitable route, including via inhalation, topically, nasally, orally, parenterally, or rectally.

Typical formulations for topical administration include creams, ointments, sprays, lotions, and patches. The pharmaceutical composition can, however, be formulated for any type of administration, e.g., intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral, rectal, or vaginal administration is also contemplated.

### 2. Routes of Administration

Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Suitable formulations for transdermal application include an effective amount of a compound or agent of the present invention with carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

For oral administration, a pharmaceutical composition or a medicament can take the form of, for example, a tablet or a capsule prepared by conventional means with a pharmaceutically acceptable excipient. Preferred are tablets and gelatin capsules comprising the active ingredient, i.e., a BTK inhibitor or a nucleic acid encoding a BTK inhibitor, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

Compounds and agents of the present invention can be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient.

For administration by inhalation, the active ingredient, e.g., a BTK inhibitor or a nucleic acid encoding a BTK inhibitor, may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

The inhibitors can also be formulated in rectal compositions, for example, suppositories or retention enemas, for example, containing conventional suppository bases, for example, cocoa butter or other glycerides.

Furthermore, the active ingredient can be formulated as a depot preparation. Such long-acting formulations can be administered by implantation (for example, subcutaneously or intramuscularly) or by intramuscular injection. Thus, for example, the active ingredient can be formulated with suitable polymeric or hydrophobic materials (for example as an emulsion in an acceptable oil) or ion exchange resins, or as sparingly soluble derivatives, for example, as a sparingly soluble salt.

A pharmaceutical composition or medicament of the present invention comprises (i) an effective amount of a compound as described herein that decreases the level or activity of BTK protein, and (ii) another therapeutic agent. When used with a compound of the present invention, such therapeutic agent may be used individually, sequentially, or in combination with one or more other such therapeutic agents (e.g., a first therapeutic agent, a second therapeutic agent, and a compound of the present invention). Administration may be by the same or different route of administration or together in the same pharmaceutical formulation.

### 3. Dosage

Pharmaceutical compositions or medicaments can be administered to a subject at a therapeutically effective dose to prevent, treat, or control neuroblastoma as described herein. The pharmaceutical composition or medicament is administered to a subject in an amount sufficient to elicit an effective therapeutic response in the subject.

The dosage of active agents administered is dependent on the subject's body weight, age, individual condition, surface area or volume of the area to be treated and on the form of administration. The size of the dose also will be determined by the existence, nature, and extent of any adverse effects that accompany the administration of a particular compound in a particular subject. For example, each type of BTK inhibitor or nucleic acid encoding a BTK inhibitor will likely have a unique dosage. A unit dosage for oral administration to a mammal of about 50 to 70 kg may contain between about 5 and 500 mg of the active ingredient. Typically, a dosage of the active compounds of the present invention, is a dosage that is sufficient to achieve the desired effect. Optimal dosing schedules can be calculated from measurements of agent accumulation in the body of a subject. In general, dosage may be given once or more daily, weekly, or monthly. Persons of ordinary skill in the art can easily determine optimum dosages, dosing methodologies and repetition rates.

To achieve the desired therapeutic effect, compounds or agents may be administered for multiple days at the therapeutically effective daily dose. Thus, therapeutically effective administration of compounds to treat a pertinent condition or disease described herein in a subject requires periodic (e.g., daily) administration that continues for a period ranging from three days to two weeks or longer. Typically, agents will be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in the subject. For example, one can administer the agents every other day, every third day, or, if higher dose ranges are employed and tolerated by the subject, once a week.

Optimum dosages, toxicity, and therapeutic efficacy of such compounds or agents may vary depending on the relative potency of individual compounds or agents and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Agents that exhibit large therapeutic indices are preferred. While agents that exhibit toxic side effects can be used, care should be taken to design a delivery system that targets such agents to the site of affected tissue to minimize potential damage to normal cells and, thereby, reduce side effects.

The data obtained from, for example, cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any agents used in the methods of the invention, the therapeutically effective dose can be estimated initially from cell culture assays. A dose can be formulated in animal models to achieve a circulating plasma concentration range that includes the IC₅₀ (the concentration of the agent that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of agents is from about 1 ng/kg to 100 mg/kg for a typical subject.

Exemplary dosages for a BTK inhibitor or a nucleic acid encoding a BTK inhibitor described herein are provided. Dosage for a BTK inhibitor-encoding nucleic acid, such as an expression cassette, can be between 0.1-0.5 mg/application, with intravitreous administration (e.g., 5-30 mg/kg). Small organic compounds inhibitors can be administered orally at between 5-1000 mg, or by intravenous infusion at between 10-500 mg/ml. Monoclonal antibody inhibitors can be administered by intravenous injection or infusion at 50-500 mg/ml (over 120 minutes); 1-500 mg/kg (over 60 minutes); or 1-100 mg/kg (bolus) five times weekly. BTK protein or mRNA inhibitors can be administered subcutaneously at 10-500 mg; 0.1-500 mg/kg intravenously twice daily, or about 50 mg once weekly, or 25 mg twice weekly.

Pharmaceutical compositions of the present invention can be administered alone (e.g., one BTK inhibitor only) or in combination with at least one additional therapeutic compound (e.g., in combination with one or more known chemotherapy agents for treating neuroblastoma). Exemplary advantageous therapeutic compounds may also include systemic and topical anti-inflammatories, pain relievers, anti-histamines, anesthetic compounds, and the like. The additional therapeutic compound can be administered at the same time as, or even in the same composition with, main active ingredient (e.g., a BTK inhibitor or a nucleic acid encoding a BTK inhibitor). The additional therapeutic compound can also be administered separately, in a separate composition, or a different dosage form from the main active ingredient. Some doses of the main ingredient, such as a BTK inhibitor or a nucleic acid encoding a BTK inhibitor, can be administered at the same time as the additional therapeutic compound, while others are administered separately, depending on the particular symptoms and characteristics of the individual.

The dosage of a pharmaceutical composition of the invention can be adjusted throughout treatment, depending on severity of symptoms, frequency of recurrence, and physiological response to the therapeutic regimen. Those of skill in the art commonly engage in such adjustments in therapeutic regimen.

### IV. Kits

The invention provides compositions and kits for practicing the methods described herein to treat neuroblastoma by using a BTK inhibitor to suppress BTK expression and/or activity. The compositions may be used to treat patients who have received a diagnosis of the disease and may have been treated by conventional methods, e.g., by surgery, chemotherapy, and/or radiotherapy.

Kits for carrying out suppression of BTK mRNA or protein level may include at least one oligonucleotide (e.g., antisense oligonucleotide or siRNA) useful for specific hybridization with at least one segment of the BTK coding sequence or its complementary sequence, leading to the reduction of BTK mRNA level and subsequently the protein level of BTK. Optionally, two or more such oligonucleotides are included in the kit. In some cases, the kits may include an expression cassette that directs the expression of the oligonucleotide (e.g, in the form of a plasmid or a viral-based construct) upon being introduced into a patient's body.

Kits for carrying out suppression of BTK biological activity level may include at least one antibody useful for specific binding to the BTK protein amino acid sequence and rendering the protein inactive, as determined by reduced or abolished BTK-ALK interaction and/or reduced or abolished ERK activation that is normally mediated by BTK. The antibody can be either a monoclonal antibody or a polyclonal antibody. One exemplary BTK inhibitor is 1-((R)-3-(4-amino-3-(4-phenoxyphenyl)-1H-pyrazolo[3,4-d]pyrimidin-1-yl)pi-peridin-1-yl)prop-2-en-1-one (Ibrutinib). Optionally, a second agent for treating neuroblastoma is included in the kit. Exemplary agents include but are not limited to those named in this disclosure. In some cases, the kits may include at least two different inactivating antibodies that specific bind to the BTK protein and render it inactive.

Typically, the kits include an appropriate amount of one or more BTK inhibitors, optionally with a second therapeutic agent against neuroblastoma. The inhibitor(s) and agent are often packaged in multiple-dose packaging for ease of use. The BTK inhibitor(s) and the second therapeutic agent may be kept in the same container or in separate containers. In addition, the kits of this invention may provide instruction manuals to guide users in the proper application of the BTK inhibitor(s).

### EXAMPLES

The following examples are provided by way of illustration only and not by way of limitation. Those of skill in the art will readily recognize a variety of non-critical parameters that could be changed or modified to yield essentially the same or similar results.

### Introduction

BTK is a tyrosine kinase that is expressed in hematopoietic cells and plays a key role in BCR signaling. It is disclosed herein the discovery of BTK expression, its interaction with ALK and its contribution of ERK activation in neuroblastoma. Importantly, it has been shown that BTK inhibitor suppresses proliferation and survival of neuroblastoma cells in vitro and reduces neuroblastoma growth in vivo. Hence, it has been demonstrated that BTK is a therapeutic target of neuroblastoma, thus providing a new class of therapeutic agents for neuroblastoma. For instance, this invention provides a method of treating neuroblastoma with an inhibitor of BTK, 1-((R)-3-(4-amino-3-(4-phenoxyphenyl)-1H-pyrazolo[3,4-d]pyrimidin-1-yl)pi-peridin-1-yl)prop-2-en-1-one (Ibrutinib). This inhibitor and pharmaceutically acceptable compositions thereof are useful for treating neuroblastoma, alone or in combination with other therapeutic agents.

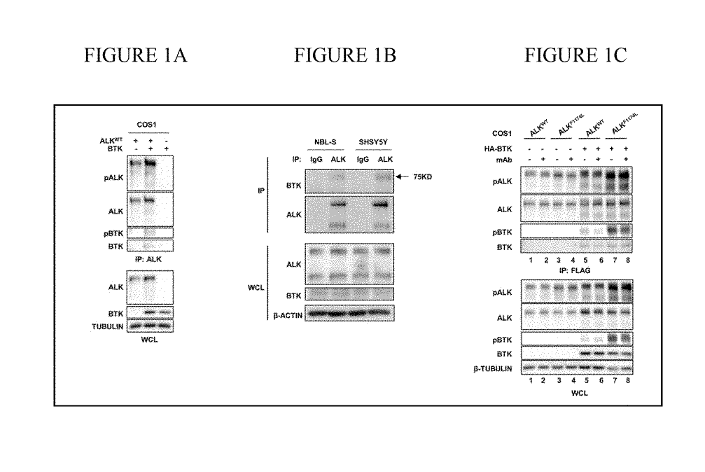

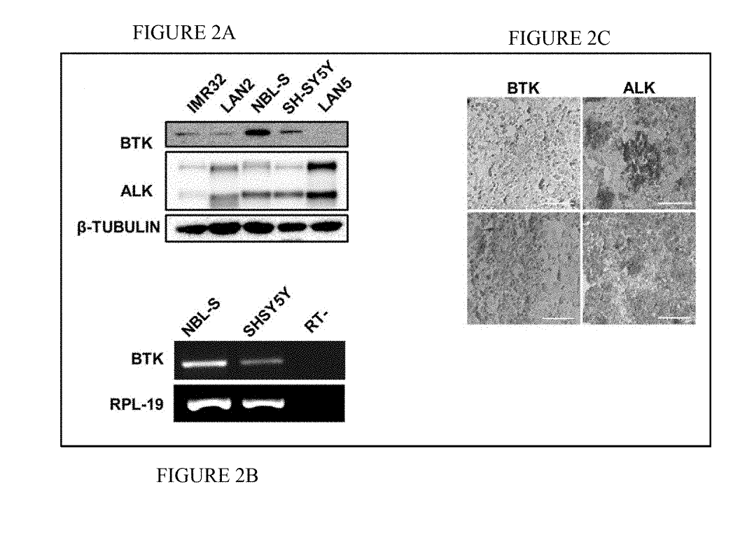

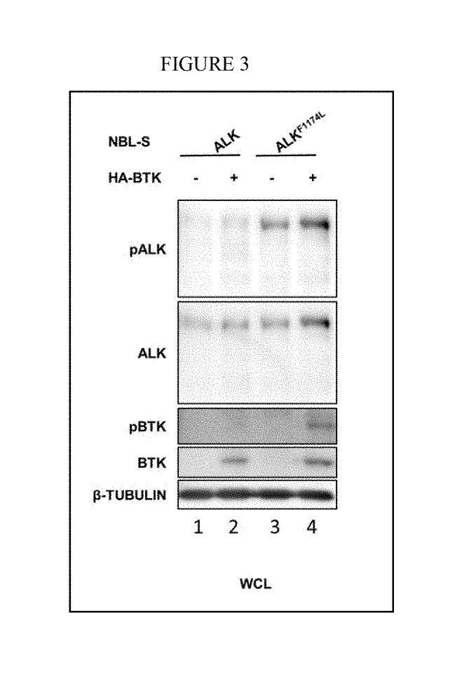

In relation to the use of BTK inhibitors for the treatment of patients who have been diagnosed with neuroblastoma, this disclosure provides information regarding the expression of BTK in neuroblastoma (FIGS. 1A-1C). This disclosure also demonstrates that BTK small hairpin RNA knockdowns and a small molecule inhibitor Ibrutinib can each decrease the proliferation and survival of neuroblastoma cell lines, and that the small molecule inhibitor can be used to inhibit the growth of neuroblastomas in mouse models (FIGS. 2A-2C and FIG. 3).

### Materials and Methods

### Patient Samples, Cell Lines, and Reagents

The PI3 kinase inhibitor GDC0941, BTK inhibitor Ibrutinib, ALK inhibitor Crizotinib and NVP-TAE-684 were purchased from ADOOQ. The cycloheximide and aprotinin were ordered from Sigma.

The antibodies against phospho-ERK1/2, phospho-STAT3, phospho-AKT, BTK, ALK, STAT3, ERK, PARP, and Cleaved-Caspase 3, were purchased from Cell Signaling Technology (USA). ALK activating antibody mAb16-39 was from Immuno-Biological Laboratories Co. (Japan). Other antibodies used in this research are listed as following, HA (Millipore), FLAG and β-actin (Sigma), and β-TUBULIN (Abcam).

### Cell Culture

Neuroblastoma cell lines NBL-S, SH-SY5Y, IMR32, LAN2 and LAN5 were purchased from DSMZ (Germany) and cultured as recommended. EcoPack virus packaging cell and COS1 cell were grown in Dulbecco's modified Eagle's medium (Hyclone) supplemented with 10% fetal bovine serum, 100 units/ml penicillin and 100 μg/ml streptomycin. Ba/F3 cells were grown in RPMI 1640 medium supplemented with 10% heat-inactivated fetal bovine serum (Hyclone), 100 units/ml penicillin and 100 μg/ml streptomycin, and 10 ng/ml recombinant murine IL-3.

To establish Ba/F3 cells stably expressing ALK, the pMSCV-ALK^{WT} and pMSCV-ALK^{F1174L} were transfected into EcoPack cells respectively. Supernatants were collected to infect Ba/F3 cells followed by selection with puromycin (1.2 μg/ml). Expression of ALK^{WT} and ALK^{F1174L} were confirmed by Western blotting, respectively.

### BTK Expression

BTK cDNA was purchased from Addgene. The open reading frame (ORF) of BTK and the ORF tagged with HA were subcloned into pCS²⁺. The DNA constructs were transfected into COS1, SH-SY5Y or NBL-S cells for overexpression, respectively.

### Immunoprecipitation and Western Blotting

The immunoprecipitation was performed as previously published (11, 12). Briefly, cells were washed in PBS and then lysed in a lysis buffer containing 1% Triton X-100, 150 mM NaCl, 25 mM Tris, pH 7.5, 5 mM EDTA, 10% glycerol, 1 mM Na₃VO₄, 2 μg/ml aprotinin and 1 mM phenylmethylsulfonyl fluoride. The lysates were centrifuged at 14000×g for 10 minutes at 4° C. and supernatants were incubated with indicated antibody for 1 hour followed by incubation with Dynabeads protein G (Life Technologies) for 30 minutes at 4° C. The immunoprecipitates were separated by SDS-PAGE and transferred to Immobilon P membranes (Millipore). Membranes were blocked with 0.1% Tween-20 in PBS for 1 hour at room temperature followed by incubation with desired antibody overnight at 4° C. After washing with 0.05% Tween-20 in PBS, membranes were incubated with secondary horseradish peroxidase-conjugated antibody for 1 hour at room temperature and washed with 0.05% Tween 20 in PBS. The immunodetection was performed by Millipore ECL reagent.

### Proliferation Assays

Neuroblastoma cells were seeded in 96-well plates at a concentration of 5×10³ per well. After siRNA transfection or inhibitor treatment for 48 hours, 10 ul MTT (5 μg/L) is added into every well followed by incubation for 4 hours. The absorbance was measured at A570 nm.

### Cell Cycle

The cultured neuroblastoma cells were trypsinized, washed with ice-cold PBS, and then fixed with 70% ethanol overnight at −20° C. The fixed cells were then washed with PBS and re-suspended in DNA extraction buffer (0.192 M Na₂HPO₄, 4 mM Citric acid, pH 7.8). After spin down and resuspension in the staining buffer (PBS with 0.1% triton X-100 (v/v), 20 mM PI, 10 μg/L RNase), cells were analyzed by flow cytometer.

### Apoptosis Assay

For cell apoptosis assay, the neuroblastoma cells were trypsinized and stained with cell apoptosis kit (Roche) according to the manufacturer's instruction. Survived cells and apoptotic cells were calculated by flow cytometer.

Statistical analysis was performed by using GraphPad Prism 5.0. The results were presented as mean values±standard deviation (S.D.) of separate experiments (n≥3). Data for multiple variable comparisons were analyzed by one-way analysis of variance (ANOVA). The statistical significance level was set as *P<0.05 and **P<0.01.

### Results

### BTK is a Novel Interaction Partner of ALK

In this study, the inventors performed a large-scale immunoprecipitation of ALK, followed by mass spectrometry analysis to identify the ALK interaction partners. The Bruton's tyrosine kinase (BTK), was identified as an ALK binding partner, and the association between ALK and BTK was further confirmed by co-immunoprecipitation (Co-IP) in COS1 cells expressing the two proteins (FIG. 1A). Endogenous BTK was detected in the ALK immunoprecipitates in NBL-S and SH-SY5Y cells which harbor ALK^{WT} and ALK^{F1174L}, respectively (FIG. 1B), which further confirmed the physical interactions between the ALK and BTK in neuroblastoma cells.

Since BTK physically interacts with ALK, it was next investigated whether BTK regulates ALK expression or activation. ALK^{WT} or ALK^{F1174L} was expressed either separately or together with BTK in COS1 cells. Immunoprecipitation indicated again the association between ALK and BTK (FIG. 1C). In addition to the binding of two proteins, the results showed that the phosphorylation of both ALK^{WT} and ALK^{F1174L} is enhanced remarkably in the presence of BTK (FIG. 1C, lane 5 vs lane 1, lane 7 vs lane 3), and the increase of pALK^{F1174L} phosphorylation appeared in a more profound manner. Furthermore, the expression of both ALK^{WT} and ALK^{F1174L} was enhanced in the presence of BTK.

BTK is Expressed in Neuroblastoma Cells and Interacts with ALK in Neuroblastoma Cells

By using Western blot, expression of BTK was detected in neuroblastoma cell lines including IMR32, LAN2 and NBL-S and SHSY5Y, while it is very weak in LAN5 cells (FIG. 2A). In line with this finding, the expression of BTK was detected in tumor tissues from two of four neuroblastoma patients, which was validated by the positive ALK staining (FIG. 2B). The expression of BTK in NBL-S and SH-SY5Y cell lines was also detected by RT-PCR (FIG. 2C).

BTK is a tyrosine kinase and its activation requires phosphorylation of tyrosine 223 (Y223) and tyrosine 551 (Y551)(16). It is notable that overexpression of ALK^{F1174L} induced much stronger BTK phosphorylation compared to ALK^{WT} in both COS1 cells and neuroblastoma cells (FIG. 1C, lane 7 vs lane 5; FIG. 3, lane 4 vs lane 2).

### Ibrutinib Inhibited ERK Phosphorylation

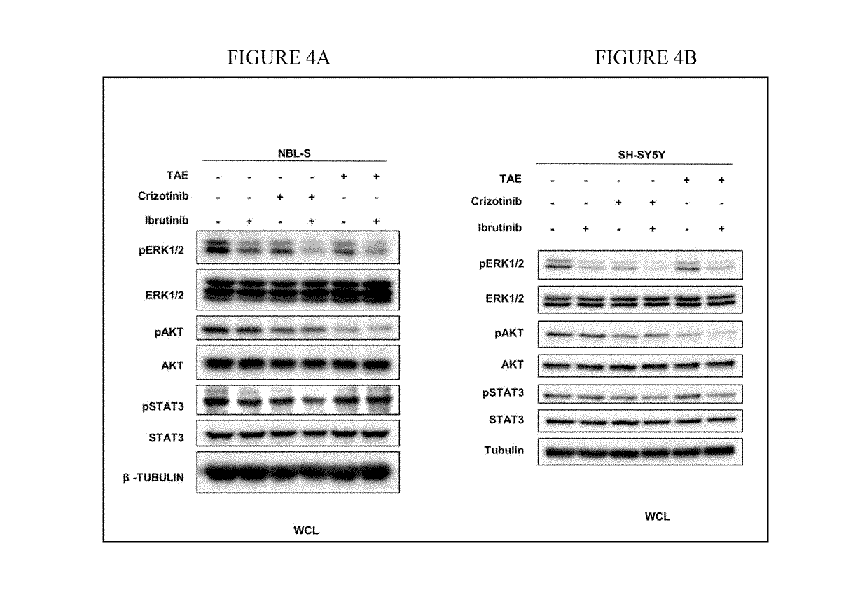

Treatment of both NBL-S and SH-SY5Y cell lines with BTK inhibitor Ibrutinib inhibited ERK activation (lane 2 in FIGS. 4A and B). In addition, ALK inhibitors NVP-TAE684 and Crizotinib further enhanced the inhibition of ERK phosphorylation caused by BTK inhibitor Ibrutinib (FIGS. 4A and B).

### BTK Regulates Proliferation of Neuroblastoma Cells

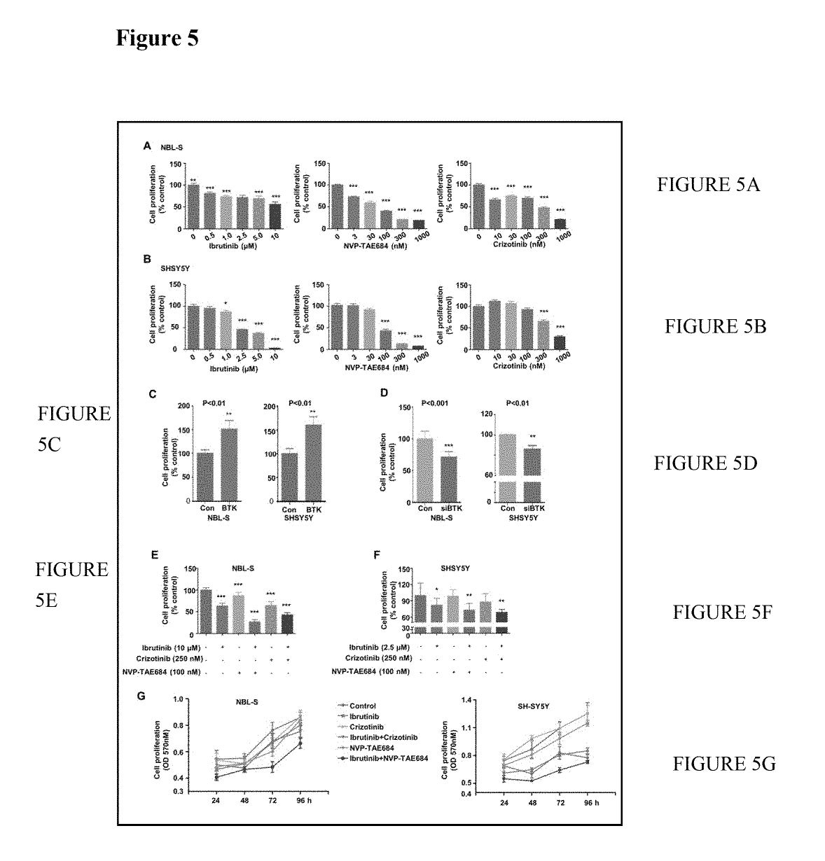

The role of BTK in the cell proliferation of neuroblastoma cells was tested. Treatment of neuroblastoma cells NBL-S cells and SH-SY5Y with BTK inhibitor, Ibrutinib, inhibited cell proliferation (FIGS. 5A and B). As expected, the proliferation of both cells can be inhibited by ALK inhibitors Crizotinib and NVP-TAE684, although SH-SY5Y cells are insensitive to Crizotinib, which could be explained by the resistance of ALK^{F1174L} to Crizotinib (FIGS. 5A, B). Overexpression and knockdown of BTK in both cell lines respectively enhanced or reduced cell proliferation, which further confirms that BTK contributes to the proliferation of neuroblastoma cells (FIGS. 5C, D). Use of both ALK and BTK inhibitors further inhibited proliferation of neuroblastoma cells (FIGS. 5E,F). MTT assay was next performed at different time points to measure the cell proliferation rates upon the separate treatment or combination of the inhibitors. All three inhibitors can reduce the cell proliferation. Addition of Ibrutinib caused a much stronger inhibition of cell proliferation in both NBL-S and SH-SY5Y cells compared to separate treatment of Crizotinib or NVP-TAE684 (FIG. 5G), indicating a potential combination use of both ALK and BTK inhibitors in the treatment of neuroblastoma.

### Inhibition of BTK Induces Cell Cycle Arrest and Promotes Cell Apoptosis of Neuroblastoma Cells.

Cell cycle and cell survival were further analyzed in order to delineate the role of BTK in the oncogenesis of neuroblastoma. It has been reported that ALK inhibitors NVP-TAE684 and Crizotinib induced G0/G1 cell cycle arrest and increased cell apoptosis in neuroblastoma cells (17, 18). Cell cycle distribution of SH-SY5Y cells was measured upon the inhibitor treatment. Ibrutinib treatment apparently resulted in more cells arrested in G0/G1 phase compared to control cells as did by NVP-TAE684. As expected, Crizotinib did not obviously increase the G0/G1 portion of SH-SY5Y cells at the tested concentration.

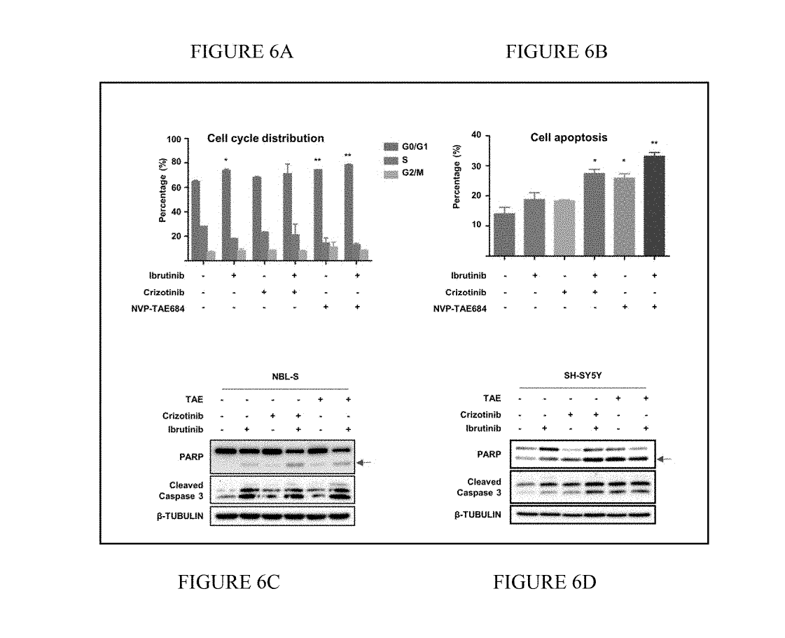

The combined treatment with Ibrutinib and NVP-TAE684 further increase cell portion at G0/G1 phase to 78.3%, compared with the control (64.9%) and single inhibitor treatment, e.g. ibrutinib (73.8%), NVP-TAE684 (74.5%) and Crizotinib (68.1%). However, the combination of Ibrutinib and Crizotinb did not obviously increase the ratio of G0/G1 phase compared the separate treatment of Ibrutinib (66.1% vs. 64.5%) (FIG. 6A).

Flow cytometry assay indicated that Ibrutinib treatment increased the cell apoptosis ratio of SH-SY5Y as did Crizotinib and NVP-TAE684 (FIG. 6B). In line with this assay, cleaved PARP and caspase 3, two markers of cell apoptosis (19), were induced by Ibrutinib, which was further enhanced by addition of ALK inhibitors (FIGS. 6C, D).

### Ibrutinib Inhibits Growth of Neuroblastoma Xenografts in Nude Mice

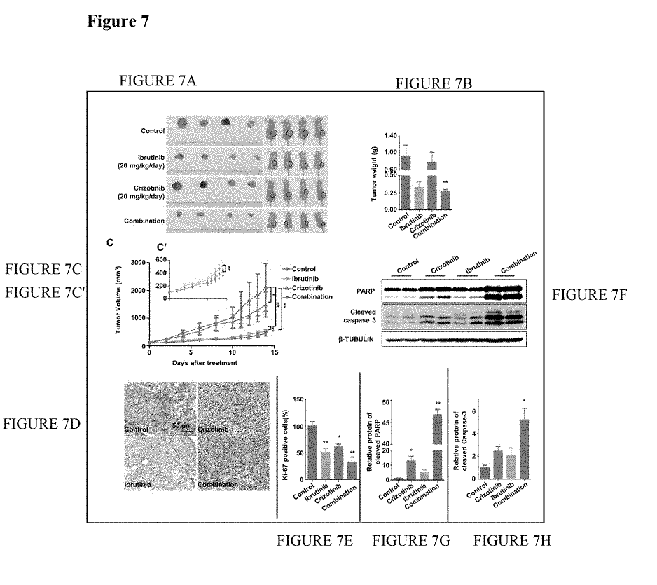

BTK inhibitor Ibrutinib is currently being used to treat B-cell malignances (20). Crizotinib exhibits remarkable clinical activity in targeting NSCLC. However, its efficacy as a single drug could be dramatically reduced because of the resistance of ALK^{F1174L} mutant in neuroblastoma. This study indicates that Ibrutinib treatment causes cell death and inhibits cell proliferation of neuroblastoma cells in vitro, it was therefore further tested: 1) whether Ibrutinib can inhibit xenograft growth in nude mice; and 2) whether it can synergize the inhibitory effect of Crizotinib on neuroblastoma xenograft growth. Neuroblastoma xenografts in nude mice were established by injection of SH-SY5Y cells. When the tumor size reached 100 mm³, the mice were then administrated by intraperitoneally injection with PBS, Ibrutinib, Crizotinib and the combination of Ibrutinib and Crizotinib, respectively. Administration of BTK inhibitor Ibrutinib remarkably attenuated the growth of neuroblastoma xenograft, indicating a key role of BTK in the carcinogenesis of neuroblastoma in vivo. ALK inhibitor Crizotinib only moderately inhibited the growth of neuroblastoma xenograft, while the combination of Ibrutinib and Crizotinib moderately enhanced the tumor inhibition compared to Ibrutinib alone (FIGS. 7A-C). Immunostaining with Ki76 antibody revealed that both Crizotinib and Ibrutinib inhibited proliferation of neuroblastoma cells in vivo, and the combination of Crizotinib and Ibrutinib caused an even stronger inhibition of cell proliferation (FIGS. 7D,E). Moreover, both Crizotinib and Ibrutinib potentiated cell apoptosis in tumor xenograft revealed by increase of cleaved caspase 3 and PARP, and combination of two inhibitors induced stronger apoptosis (FIGS. 7F-H). Thus, the xenograft assay strongly supported the in vitro data, indicating that Ibrutinib inhibits cell proliferation, concomitantly elevates apoptosis in neuroblastoma cells.

Taken all together, BTK is identified as a novel interaction partner of ALK. BTK inhibitor, Ibrutinib inhibits neuroblastoma cell proliferation, and induces cell apoptosis. Administration of Ibrutinib attenuates the growth of tumor xenograft induced by SH-SY5Y, indicating therapeutic potentials for treating ALK positive neuroblastoma.

### Discussion

This study provided the first evidence of BTK's direct involvement in neuroblastoma through cellular signaling mediated by ERK activation. Thus, a novel therapeutic strategy is provided by way of targeting BTK, e.g., by using one or more BTK inhibitors.

The aberrant activation of ALK is one of the major oncogenic drivers for malignancies including NSCLC, ALCL, IMT, as well as neuroblastoma (21, 22). ALK fusion genes induced by gene rearrangement mainly occur in NSCLC, ALCL, IMT and DLBCL, whereas point mutations of ALK are frequently found in neuroblastoma with few reports in thyroid and lung cancers (22). ALK inhibitor Crizotinib has been used in the treatment of NSCLC and it has dramatically improved the treatment outcome of NSCLC (23). Moreover, the FDA proved Ceritinib and Alectinib as second line treatment for the relapsed (22). In contrary to the treatment of NSCLC, treatment of neuroblastoma with ALK inhibitors displayed disappointing efficacy (24, 25). Although both ALK fusion proteins and point mutations can induce ligand independent activation of ALK, the possible differential mechanisms of activation between the two categories of ALK mutants could change efficacy of ALK inhibitors in treating these two cancers. In order to better understand the signal transduction of ALK in neuroblastoma, cell lines that stably express ALK^{WT} or ALK^{F1174L} have been established, and BTK has been identified as a novel interaction partner of ALK.

BTK has been widely studied in B cell development and B cell malignancies. It is conventionally considered as a tyrosine kinase expressed hematopoietic cells (16, 26). However, some studies suggest that BTK is also expressed in other organs and plays oncogenic roles in prostate cancer and colon cancer (27, 28). In addition to these findings, this study reveals that BTK is expressed in neuroblastoma, further extending the implication of BTK in non-hematopoietic malignancies.

Ibrutnib could potentially be used for treating neuroblastoma harboring ALK mutations. The effects of Ibrutinib were tested in xenograft assay. It reduced the growth of tumor xenograft induced by SH-SY5Y cells in nude mice, displaying much stronger efficacy compared to Crizotinib (FIGS. 4A-C). Furthermore, addition of ALK inhibitor Crizotinib leads to further attenuation of tumor growth, suggesting a potential use of Ibrutinib or the combination of BTK inhibitor and ALK inhibitor in the treatment of ALK positive neuroblastoma. Ibrutinib is currently used for treatment of chronic lymphocytic leukemia, Mantle cell lymphoma and Waldenstrom's macroglobulinemia (29, 30), and the data generated in this study support the use of Ibrutinib for neuroblastoma.

Taken all together, BTK expression was identified in neuroblastoma cells. BTK interacts with ALK and can cause the decrease of ALK ubiquitination, therefore increases the stability of ALK. Furthermore, BTK contributes to the oncogenesis of neuroblastoma. It is noted that the BTK inhibitor, Ibrutinib, can inhibit the growth of neuroblastoma xenograft in nude mice, and the combined administration with ALK inhibitor Crizotinib can further enhance the inhibition, which provide experimental evidence supporting repurposing Ibrutinib to treat neuroblastoma. This study shed lights on the complexity of BTK function and indicates an important oncogenic role of BTK in neuroblastoma.

All patents, patent applications, and other publications, including GenBank Accession Numbers, cited in this application are incorporated by reference in the entirety for all purposes.

### REFERENCES

1. Park J R, Eggert A, and Caron H. Neuroblastoma: biology, prognosis, and treatment. Hematol Oncol Clin North Am. 2010; 24(1):65-86.
2. Louis C U, and Shohet J M. Neuroblastoma: molecular pathogenesis and therapy. Annu Rev Med. 2015; 66:49-63.
3. Kurosaki T. Functional dissection of BCR signaling pathways. Curr Opin Immunol. 2000; 12(3):276-81.
4. Schaeffer E M, Broussard C, Debnath J, Anderson S, McVicar D W, and Schwartzberg P L. Tec family kinases modulate thresholds for thymocyte development and selection. J Exp Med. 2000; 192(7):987-1000.
5. Horwood N J, Mahon T, McDaid J P, Campbell J, Mano H, Brennan F M, et al. Bruton's tyrosine kinase is required for lipopolysaccharide-induced tumor necrosis factor alpha production. J Exp Med. 2003; 197(12):1603-11.
6. Iwaki S, Tkaczyk C, Satterthwaite A B, Halcomb K, Beaven M A, Metcalfe D D, et al. Btk plays a crucial role in the amplification of Fc epsilonRI-mediated mast cell activation by kit. J Biol Chem. 2005; 280(48):40261-70.
7. Vassilev A, Ozer Z, Navara C, Mahajan S, and Uckun F M. Bruton's tyrosine kinase as an inhibitor of the Fas/CD95 death-inducing signaling complex. J Biol Chem. 1999; 274(3):1646-56.
8. Quek L S, Bolen J, and Watson S P. A role for Bruton's tyrosine kinase (Btk) in platelet activation by collagen. Current biology: CB. 1998; 8(20):1137-40.
9. Iwaki S, Kihara A, Sano T, and Igarashi Y. Phosphorylation by Pho85 cyclin-dependent kinase acts as a signal for the down-regulation of the yeast sphingoid long-chain base kinase Lcb4 during the stationary phase. J Biol Chem. 2005; 280(8):6520-7.
10. Peng J B, Chen X Z, Berger U V, Vassilev P M, Tsukaguchi H, Brown E M, et al. Molecular cloning and characterization of a channel-like transporter mediating intestinal calcium absorption. J Biol Chem. 1999; 274(32):22739-46.
11. Wang C, Kam R K, Shi W, Xia Y, Chen X, Cao Y, et al. The Proto-oncogene Transcription Factor Ets1 Regulates Neural Crest Development through Histone Deacetylase 1 to Mediate Output of Bone Morphogenetic Protein Signaling. J Biol Chem. 2015; 290(36):21925-38.
12. Sun J, Mohlin S, Lundby A, Kazi J U, Hellman U, Pahlman S, et al. The PI3-kinase isoform p110delta is essential for cell transformation induced by the D816V mutant of c-Kit in a lipid-kinase-independent manner. Oncogene. 2014; 33(46):5360-9.
13. Wu J, Liu C, Tsui S T, and Liu D. Second-generation inhibitors of Bruton tyrosine kinase. J Hematol Oncol. 2016; 9(1):80.
14. Li Y, Ramirez-Valle F, Xue Y, Ventura J I, Gouedard O, Mei J, et al. Population Pharmacokinetics and Exposure Response Assessment of CC-292, a Potent BTK Inhibitor, in Patients With Chronic Lymphocytic Leukemia. J Clin Pharmacol. 2017.
15. Akinleye A, Chen Y, Mukhi N, Song Y, and Liu D. Ibrutinib and novel BTK inhibitors in clinical development. J Hematol Oncol. 2013; 6:59.
16. Buggy J J, and Elias L. Bruton tyrosine kinase (BTK) and its role in B-cell malignancy. International reviews of immunology. 2012; 31(2):119-32.
17. Ding N, Li X, Shi Y, Ping L, Wu L, Fu K, et al. Irreversible dual inhibitory mode: the novel Btk inhibitor PLS-123 demonstrates promising anti-tumor activity in human B-cell lymphoma. Oncotarget. 2015; 6(17):15122-36.
18. Herman S E, Mustafa R Z, Gyamfi J A, Pittaluga S, Chang S, Chang B, et al. Ibrutinib inhibits BCR and NF-kappaB signaling and reduces tumor proliferation in tissue-resident cells of patients with CLL. Blood. 2014; 123(21):3286-95.
19. Braun F K, Mathur R, Sehgal L, Wilkie-Grantham R, Chandra J, Berkova Z, et al. Inhibition of methyltransferases accelerates degradation of cFLIP and sensitizes B-cell lymphoma cells to TRAIL-induced apoptosis. PLoS One. 2015; 10(3):e0117994.
20. Roskoski R, Jr. Ibrutinib inhibition of Bruton protein-tyrosine kinase (BTK) in the treatment of B cell neoplasms. Pharmacological research. 2016; 113(Pt A):395-408.
21. Yau N K, Fong A Y, Leung H F, Verhoeft K R, Lim Q Y, Lam W Y, et al. A Pan-Cancer Review of ALK Mutations: Implications for Carcinogenesis and Therapy. Current cancer drug targets. 2015; 15(4):327-36.
22. Holla V R, Elamin Y Y, Bailey A M, Johnson A M, Litzenburger B C, Khotskaya Y B, et al. ALK: a tyrosine kinase target for cancer therapy. Cold Spring Harbor molecular case studies. 2017; 3(1):a001115.
23. Kwak E L, Bang Y J, Camidge D R, Shaw A T, Solomon B, Maki R G, et al. Anaplastic lymphoma kinase inhibition in non-small-cell lung cancer. N Engl J Med. 2010; 363(18):1693-703.
24. Mosse Y P, Lim M S, Voss S D, Wilner K, Ruffner K, Laliberte J, et al. Safety and activity of crizotinib for paediatric patients with refractory solid tumours or anaplastic large-cell lymphoma: a Children's Oncology Group phase 1 consortium study. Lancet Oncol. 2013; 14(6):472-80.
25. Geoerger B, Schulte J, Zwaan C M, Casanova M, Fischer M, Moreno L, et al. Phase I study of ceritinib in pediatric patients (Pts) with malignancies harboring a genetic alteration in ALK (ALK plus): Safety, pharmacokinetic (PK), and efficacy results. J Clin Oncol. 2015; 33(15).
26. Smith C I. From identification of the BTK kinase to effective management of leukemia. Oncogene. 2016.
27. Guo W, Liu R, Bhardwaj G, Yang J C, Changou C, Ma A H, et al. Targeting Btk/Etk of prostate cancer cells by a novel dual inhibitor. Cell death & disease. 2014; 5:e1409.
28. Grassilli E, Pisano F, Cialdella A, Bonomo S, Missaglia C, Cerrito M G, et al. A novel oncogenic BTK isoform is overexpressed in colon cancers and required for RAS-mediated transformation. Oncogene. 2016.
29. Chakraborty R, Kapoor P, Ansell S M, and Gertz M A. Ibrutinib for the treatment of Waldenstrom macroglobulinemia. Expert review of hematology. 2015; 8(5):569-79.
30. de Claro R A, McGinn K M, Verdun N, Lee S L, Chiu H J, Saber H, et al. FDA Approval: Ibrutinib for Patients with Previously Treated Mantle Cell Lymphoma and Previously Treated Chronic Lymphocytic Leukemia. Clinical cancer research: an official journal of the American Association for Cancer Research. 2015; 21(16):3586-90.

What is claimed is:

1. A method for treating neuroblastoma, comprising the step of administering to a subject in need thereof an effective amount of a Bruton's tyrosine kinase (BTK) inhibitor.

2. The method of claim 1, wherein the inhibitor is 1-((R)-3-(4-amino-3-(4-phenoxyphenyl)-1H-pyrazolo[3,4-d]pyrimidin-1-yl)pi-peridin-1-yl)prop-2-en-1-one (Ibrutinib).

3. The method of claim 1, wherein the inhibitor is a neutralizing antibody of BTK.

4. The method of claim 1, wherein the inhibitor is an antisense oligonucleotide or siRNA that suppresses BTK expression.

5. The method of claim 1, wherein the subject is co-administered with a second therapeutic agent for treating neuroblastoma.

6. The method of claim 5, wherein the second therapeutic agent is ALK inhibitor Crizotinib.

7. The method of claim 1, wherein the subject has wild-type ALK gene.

8. The method of claim 1, wherein the subject has a mutated ALK gene or has overexpression or over-activation of ALK.

9. A composition for treating neuroblastoma comprising an effective amount of a BTK inhibitor and a physiologically acceptable excipient.

10. The composition of claim 9, wherein the inhibitor is 1-((R)-3-(4-amino-3-(4-phenoxyphenyl)-1H-pyrazolo[3,4-d]pyrimidin-1-yl)pi-peridin-1-yl)prop-2-en-1-one (Ibrutinib) or a neutralizing antibody of BTK or an antisense oligonucleotide or siRNA that suppresses BTK expression.

11. The composition of claim 9, further comprising ALK inhibitor Crizotinib.

12. A method for identifying a BTK inhibitor, comprising the steps of:
(a) contacting a cell expressing both BTK and ALK with a candidate compound;
(b) determining BTK-ALK association level in the cell in step (a);
(c) comparing the BTK-ALK associate level obtained in step (b) with a control BTK-ALK association level in a control cell, which is identical to the cell in step (a) but has not been contacted with the candidate compound, and
(d) identifying the candidate compound as a BTK inhibitor, when the BTK-ALK associate level obtained in step (b) is lower than the control BTK-ALK association level.

13. The method of claim 12, wherein the BTK-ALK associate level obtained in step (b) is at least 10%, 20%, or 50% lower than the control BTK-ALK association level.

14. The method of claim 12, wherein the cell is a neuroblast.

15. The method of claim 12, further comprising, subsequent to step (d), contacting neuroblastoma cells with the candidate compound and measuring proliferation rate or apoptosis rate of the cells.

16. A kit for treating neuroblastoma in a subject, comprising a first container containing a BTK inhibitor and a second container containing a second therapeutic agent for treating neuroblastoma.

17. The kit of claim 16, wherein the BTK inhibitor is 1-((R)-3-(4-amino-3-(4-phenoxyphenyl)-1H-pyrazolo[3,4-d]pyrimidin-1-yl)pi-peridin-1-yl)prop-2-en-1-one (Ibrutinib) or a neutralizing antibody of BTK or an antisense oligonucleotide or siRNA that suppresses BTK expression.

18. The kit of claim 16, wherein the second therapeutic agent is ALK inhibitor Crizotinib.

19. The kit of claim 16, further comprising an instruction manual.
